(19) United States
(12) Patent Application Publication
(10) Pub. No.: US 2023/0181508 A1
(43) Pub. Date: Jun. 15, 2023

(54) COMPOSITIONS AND METHODS FOR LEVODOPA DELIVERY

(71) Applicant: Purdue Pharma L.P., Stamford, CT (US)

(72) Inventors: Haiyong Hugh Huang, Princeton Junction, NJ (US); Manjunath S. Shet, White Plains, NY (US)

(21) Appl. No.: 18/017,767

(22) PCT Filed: Aug. 30, 2021
(86) PCT No.: PCT/US2021/048218

§ 371 Date: Jan. 24, 2023

Related U.S. Application Data
(60) Provisional application No. 63/072,661, filed on Aug. 31, 2020.

(51) Int. Cl. A61K 31/198; A61K 9/00

(52) U.S. Cl. CPC A61K 31/198; A61K 9/0043

(57) ABSTRACT

Disclosed in certain embodiments is a method of treating Parkinson's disease comprising administering to the olfactory region of the nose of a patient in need thereof a pharmaceutical composition comprising levodopa or a pharmaceutically acceptable salt thereof and a positively charged amino acid.

### CROSS REFERENCE TO RELATED APPLICATIONS

The present application claims priority to U.S. provisional application No. 63/072,661 filed on Aug. 31, 2020, the entire contents of which are incorporated herein in its entirety. All references cited herein are hereby incorporated by reference.

### FIELD OF THE INVENTION

The invention relates to pharmaceutical compositions suitable for nasal administration. In certain embodiments, the invention relates to a pharmaceutical composition comprising levodopa (i.e., L-Dopa) or a pharmaceutically acceptable salt thereof for treating nervous system disorders (e.g., Parkinson's disease) through nasal administration.

### BACKGROUND OF THE INVENTION

Parkinson's disease is a progressive nervous system disorder that affects movement of a patient. In Parkinson's disease, neurons in the brain gradually break down or die. It is believed that many of the symptoms are due to a loss of neurons that produce dopamine, a chemical messenger in the brain. (Parkinson's Disease, Mayfield Brain & Spine, p. 1, April 2018). When dopamine levels decrease, it causes abnormal brain activity, leading to impaired movement and other symptoms of Parkinson's disease. Symptoms start gradually, sometimes starting with a barely noticeable tremor in just one hand. Tremors are common, but the disorder also commonly causes stiffness or slowing of movement. Although Parkinson's disease cannot be cured at this moment, pharmacological treatment may significantly improve symptoms.

Levodopa was approved to treat Parkinson's disease over 50 years ago and today it remains the primary treatment. Levodopa (also known as “L-Dopa”) is in a class of medications referred to as dopaminergic antiparkinsonism agents and works by being converted to dopamine in the brain. It is commonly co-administered with a decarboxylase inhibitor, such as carbidopa, to limit the proportion of the dose converted to dopamine outside of the brain and to prevent the levodopa from being broken down before it reaches the brain. When taking orally, the absorption of levodopa occurs in the upper small intestine, and levodopa's pharmacological activity is impaired by unfavorable absorption kinetics.

There exists a need in the art for a method of treating Parkinson's disease and a corresponding pharmaceutical composition that avoids the unfavorable absorption kinetics of standard oral therapy of levodopa.

### OBJECTS AND SUMMARY OF THE INVENTION

It is an object of certain embodiments of the invention to provide a pharmaceutical composition for treating Parkinson's disease.

It is an object of certain embodiments of the invention to provide a method of treating Parkinson's disease through nasal administration of a pharmaceutical composition of the disclosure.

It is an object of certain embodiments of the invention to provide a pharmaceutical composition and method thereof that avoids the unfavorable absorption kinetics associated with oral levodopa therapy.

It is an object of certain embodiments of the invention to provide a system comprising a nasal administration device containing the pharmaceutical compositions disclosed herein.

It is an object of certain embodiments of the invention to provide a method of manufacturing the pharmaceutical compositions and systems disclosed herein.

The above objects and others may be achieved by the invention which in certain embodiments is directed to a method of treating Parkinson's disease comprising administering to the olfactory region of the nose of a patient in need thereof a pharmaceutical composition comprising a therapeutically effective amount of levodopa or a pharmaceutically acceptable salt thereof, and at least one positively charged amino acid. In certain embodiments, the levodopa and the positively charged amino acid form a complex.

In certain embodiments, the invention is directed to a pharmaceutical composition comprising a therapeutically effective amount of levodopa or a pharmaceutically acceptable salt thereof, a positively charged amino acid, and a pharmaceutically acceptable nasal vehicle, wherein the pH value of the pharmaceutical composition is from about 7 to about 8.

In certain embodiments, the invention is directed to a pharmaceutical composition comprising a therapeutically effective amount of levodopa or a pharmaceutically acceptable salt thereof, a positively charged amino acid and a pharmaceutically acceptable nasal vehicle. In certain embodiments, the molar ratio of the positively charged amino acid to the levodopa or pharmaceutically acceptable salt thereof is greater than 2:1.

In certain embodiments, the invention is directed to a system comprising a device adapted to deliver a payload to the olfactory region of a human nose and a nasal composition wherein the nasal composition comprises a therapeutically effective amount of levodopa or a pharmaceutically acceptable salt thereof, at least one positively charged amino acid and a pharmaceutically acceptable nasal vehicle.

In certain embodiments, the invention is directed to a method of delivering levodopa or a pharmaceutically acceptable salt thereof to a patient identified as in need of levodopa therapy, comprising administering to the olfactory region of the nose of the patient (e.g., a human) a pharmaceutical composition comprising levodopa or pharmaceutically acceptable salt thereof and a positively charged amino acid, wherein said method selectively delivers a therapeutically effective amount of the levodopa or pharmaceutically acceptable salt thereof to the brain tissues of the patient.

In certain embodiments, the invention is directed to a process for manufacturing a pharmaceutical composition as disclosed herein comprising combining in any order, levodopa or a pharmaceutically acceptable salt thereof, a positively charged amino acid and a nasally acceptable vehicle.

In certain embodiments, the invention is directed to a process for manufacturing a system comprising containing a pharmaceutical composition as disclosed herein in a device adapted to deliver a payload to the olfactory region of a human nose.

### BRIEF DESCRIPTION OF THE DRAWINGS

The above and other features of the present invention, their nature, and various advantages will become more apparent post consideration of the following detailed description, taken in conjunction with the accompanying drawings, in which:

FIG. 1 depicts individual plasma concentrations of levodopa after intranasal administration of levodopa (1.2 mg/kg) and 10% arginine in male Sprague-Dawley rats.

FIG. 2 depicts average plasma concentrations of levodopa after intranasal administration of levodopa (1.2 mg/kg) and 10% arginine in male Sprague-Dawley rats.

FIG. 3 depicts average plasma concentrations of levodopa after intranasal administration of levodopa (1.2 mg/kg) and 10% arginine in male Sprague-Dawley rats.

FIG. 4 depicts average brain tissue concentrations of dopamine after intranasal administration of levodopa (1.2 mg/kg) and 10% arginine in male Sprague-Dawley rats.

FIG. 5 depicts average plasma concentrations of levodopa after intranasal administration of levodopa (2.4 mg/kg) and 10% arginine in male Sprague-Dawley rats.

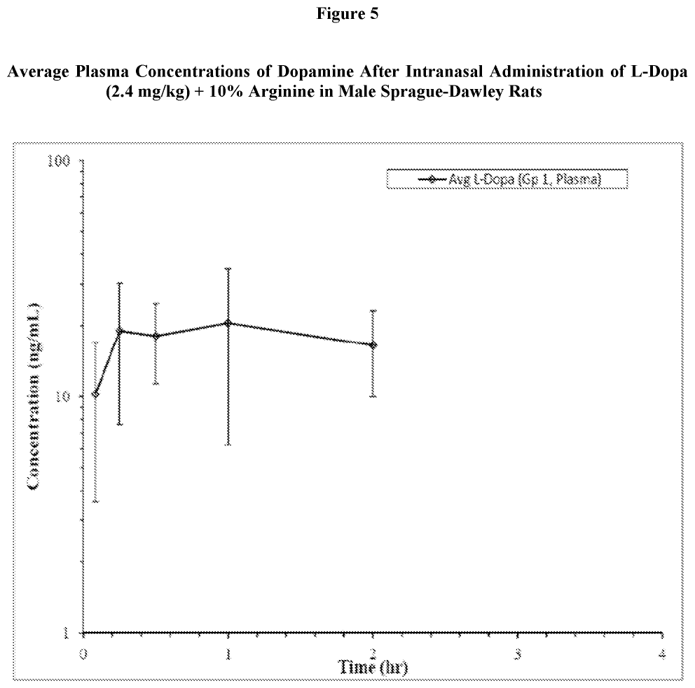

FIG. 6 depicts average brain tissue concentrations of levodopa after intranasal administration of levodopa (2.4 mg/kg) and 10% arginine in male Sprague-Dawley rats.

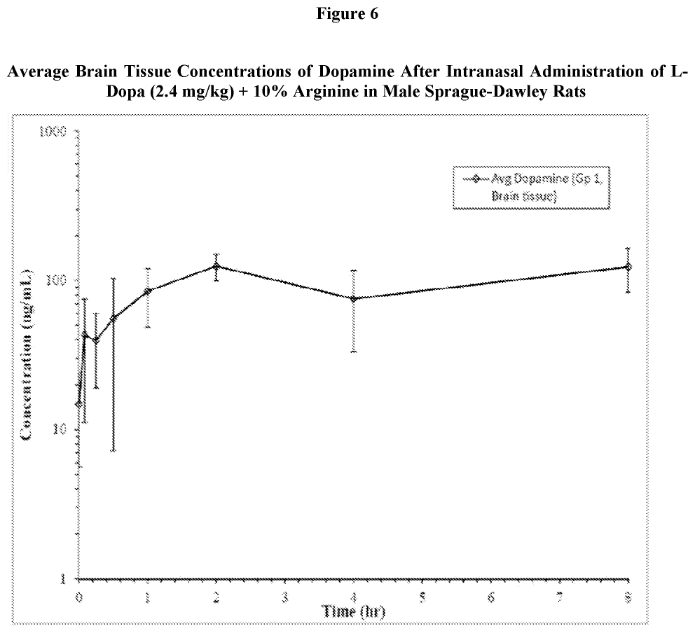

FIG. 7 depicts average plasma concentrations of levodopa after intranasal administration of levodopa (3.6 mg/kg) with 10% arginine in male Sprague-Dawley rats.

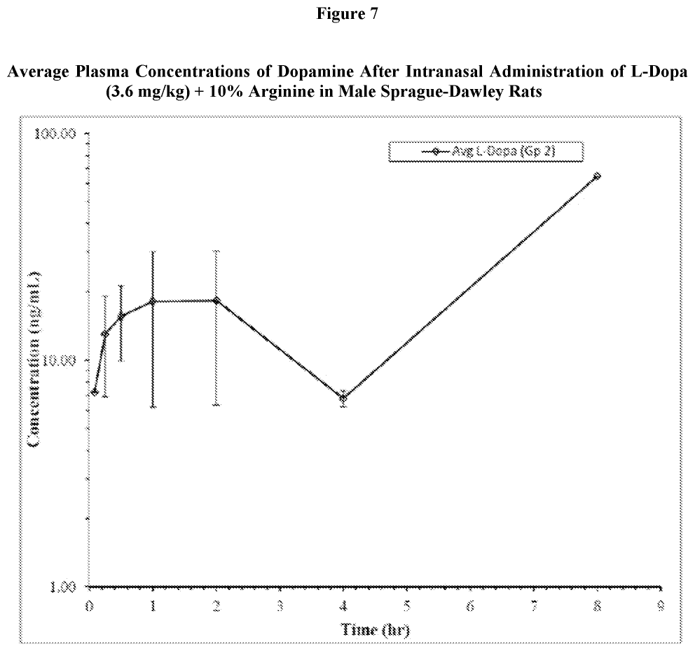

FIG. 8 depicts average brain tissue concentrations of levodopa after intranasal administration of levodopa (3.6 mg/kg) with 10% arginine in male Sprague-Dawley rats.

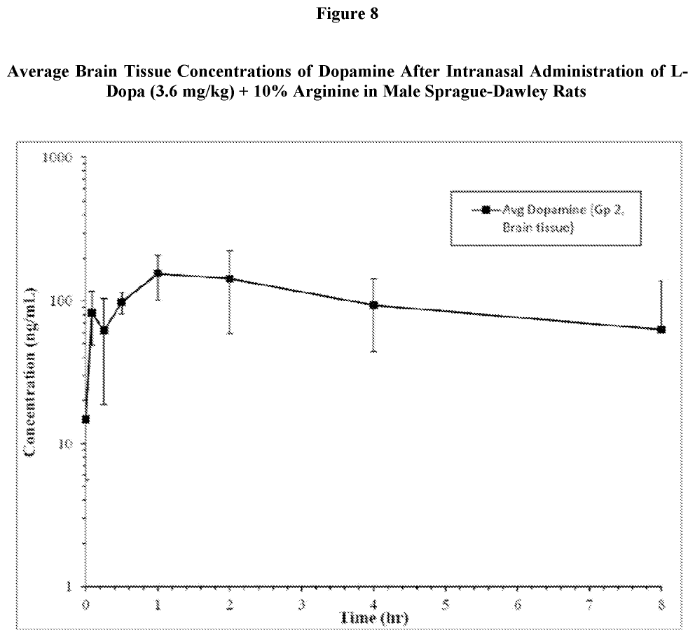

FIG. 9 depicts average plasma concentrations of levodopa after intranasal administration of levodopa (2.4 mg/kg) with 5% arginine in male Sprague-Dawley rats.

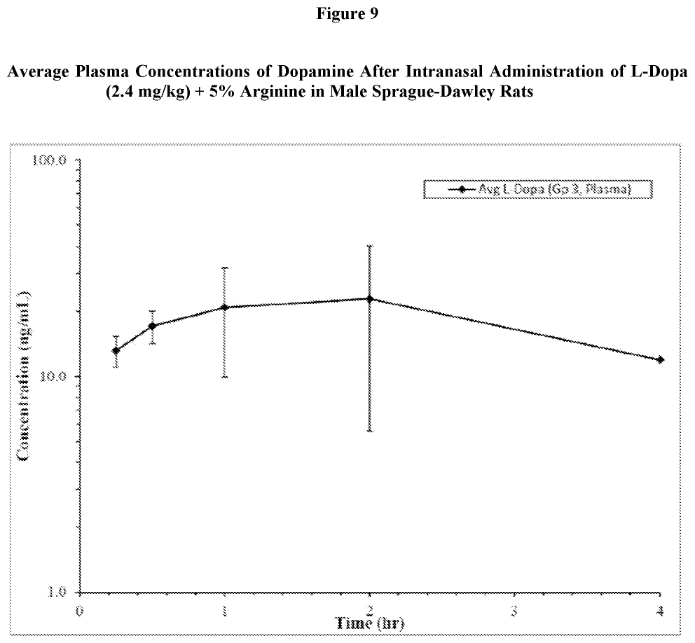

### DEFINITIONS

As used herein, the singular forms “a,” “an,” and “the” include plural references unless the context clearly indicates otherwise. Thus, for example, reference to an “excipient” includes a single excipient as well as a mixture of two or more different excipients, and the like.

As used herein, the term “about” in connection with a measured quantity or time, refers to the normal variations in that measured quantity or time, as expected by one of ordinary skill in the art in making the measurement and exercising a level of care commensurate with the objective of measurement. In certain embodiments, the term “about” includes the recited number ±10%, such that “about 10” would include from 9 to 11, or “about 1 hour” would include from 54 minutes to 66 minutes.

As used herein, the term “active agent” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates and crystalline forms thereof, where the salts, solvates and crystalline forms are pharmaceutically active.

As used herein, the terms “therapeutically effective” and an “effective amount” refer to that amount of an active agent or the rate at which it is administered needed to produce a desired therapeutic result.

The term “patient” means a subject (preferably a human) who has presented a clinical manifestation of a particular symptom or symptoms suggesting the need for treatment, who is treated preventatively or prophylactically for a condition, or who has been diagnosed with a condition to be treated.

The term “subject” is inclusive of the definition of the term “patient” and inclusive of the term “healthy subject,” which refers to an individual (e.g., a human) who is entirely normal in all respects or with respect to a particular condition.

The terms “treatment of” and “treating” include the administration of an active agent(s) to lessen the severity of a condition.

The terms “prevention of” and “preventing” include the avoidance of or delay the onset of a condition by a prophylactic administration of an active agent.

Recitation of ranges of values herein are merely intended to serve as a shorthand method of referring individually to each separate value falling within the range, unless otherwise indicated herein, and each separate value is incorporated into the specification as it is individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

The term “concurrently” as used herein means that a dose of one agent is administered prior to the end of the dosing interval of another agent. For example, a dose of nasal levodopa with a particular dosing interval is considered as concurrently administered with oral levodopa when the nasal dose is administered within the dosing interval of the oral levodopa.

The term “simultaneously” as used herein means that a dose of one agent is administered approximately at the same time as another agent, regardless of whether the agents are administered separately via the same or different routes of administration or in a single pharmaceutical composition or dosage form. For example, a dose of nasal levodopa may be administered separately from, but at the same time as, a dose of oral levodopa.

The term “sequentially” as used herein means that a dose of one agent is administered first and thereafter another dose of a same or different agent is administered second. For example, a dose of oral levodopa may be administered first, and thereafter a dose of nasal levodopa may be administered second. The subsequent administration of the second agent may be inside or outside the dosing interval of the first agent.

### DETAILED DESCRIPTION

By virtue of certain embodiments of the present invention, there is provided a method of managing the symptoms of Parkinson's disease in a patient by nasal administration of a levodopa pharmaceutical composition. In certain embodiments, the administration is directed to the olfactory region of the nose of the patient, and thus provides a route for the delivery of levodopa directly to the patient's brain, where it is metabolized to dopamine. In certain embodiments, systemic delivery of levodopa according to the method of the present invention is minimal or non-detectable, making it not necessary to co-administer a decarboxylase inhibitor. Although co-administration of a decarboxylase inhibitor is contemplated in certain embodiments of the invention.

In certain embodiments, the invention is directed to a method of treating Parkinson's disease comprising administering to the olfactory region of the nose of a subject or patient in need thereof a pharmaceutical composition comprising a therapeutically effective amount of levodopa or a pharmaceutically acceptable salt thereof and at least one positively charged amino acid. In a particular embodiment, the pharmaceutical composition contacts the olfactory nerves of the subject or patient during administration.

In certain embodiments, the invention is directed to a pharmaceutical composition comprising a therapeutically effective amount of levodopa or a pharmaceutically acceptable salt thereof and a positively charged amino acid, wherein the pH value of the pharmaceutical composition is from about 7 to about 8.

In certain embodiments, the invention is directed to a pharmaceutical composition comprising a therapeutically effective amount of levodopa or a pharmaceutically acceptable salt thereof and a positively charged amino acid, wherein the molar ratio of the positively charged amino acid to the levodopa or pharmaceutically acceptable salt thereof is greater than about 2:1.

In certain embodiments, the invention is directed to a system comprising a device adapted to deliver a payload to the olfactory region of a human nose and a nasal composition comprising a therapeutically effective amount of levodopa or a pharmaceutically acceptable salt thereof and a positively charged amino acid.

In certain embodiments of the invention, the pharmaceutical composition further comprises a nasally acceptable vehicle. The vehicle can be, e.g., an aqueous solution, an organic solvent (e.g., ethanol, propylene glycol, polyethylene glycols), a suspension, an ointment, a cream, a gel, or a combination thereof. In certain embodiments, the vehicle is an aqueous solution that contains greater than about 50% water, greater than about 60% water, greater than about 75% water; greater than about 90% water, or greater than about 95% water. In a particular embodiment, the vehicle is a saline solution.

In certain embodiments, the levodopa or pharmaceutically acceptable salt thereof is dissolved or partially dissolved in the nasally acceptable vehicle. In other embodiments, the levodopa or pharmaceutically acceptable salt(s) thereof is suspended or partially suspended in the nasally acceptable vehicle. The suspension could be either homogeneous or heterogeneous.

In other embodiments, the positively charged amino acid is dissolved or partially dissolved in the nasally acceptable vehicle. In other embodiments, the positively charged amino acid is suspended or partially suspended in the nasally acceptable vehicle.

In certain embodiments, the pharmaceutical composition comprises solid particles comprising the levodopa and the positively charged amino acid and is administered without a liquid vehicle (e.g., as a dry powder). In such an embodiment, the solid particles may comprise an excipient, including a polymer (e.g., polylactic glycolic acid).

In certain embodiments, the positively charged amino acid is lysine, arginine, histidine or a combination thereof. In a particular embodiment, the positively charged amino acid comprises arginine (also known as, L-arginine).

In certain embodiments, the pharmaceutical composition does not comprise a decarboxylase inhibitor (e.g., carbidopa). In certain embodiments, the composition does not comprise a decarboxylase inhibitor as there is a minimal amount levodopa that is delivered to systemic plasma.

In other embodiments, the pharmaceutical composition of the invention includes a decarboxylase inhibitor (e.g., carbidopa). The decarboxylase inhibitor can be administered by the same route or a different route than the levodopa. For example, the decarboxylase inhibitor can be administered orally.

In certain embodiments, the pH of the pharmaceutical composition of the invention is from about 6 to about 9; from about 6.5 to about 8.5; from about 7 to about 8; or about 7.5.

In certain embodiments, the levodopa or pharmaceutically acceptable salt thereof is present in the pharmaceutical composition at a concentration of greater than about 4 mg/mL; greater than about 6 mg/mL; greater than about 12 mg/mL; greater than about 15 mg/mL; greater than about 18 mg/mL; greater than about 22 mg/mL; from about 6 mg/mL to about 30 mg/mL; from about 15 mg/mL to about 25 mg/mL; from about 16 mg/mL to about 25 mg/mL; from about 10 mg/mL to about 20 mg/mL; from about 20 mg/mL to about 30 mg/mL; from about 6 mg/mL to about 10 mg/mL; from about 7 mg/mL to about 9 mg/mL; or about 8 mg/mL.

In certain embodiments, the levodopa or pharmaceutically acceptable salt thereof is administered in an amount of greater than about 0.5 mg/kg; greater than about 1 mg/kg; greater than about 2 mg/kg; greater than about 3 mg/kg; from about 0.5 mg/kg to about 10 mg/kg; from about 1 mg/kg to about 9 mg/kg; from about 2 mg/kg to about 8 mg/kg; from about 3 mg/kg to about 7 mg/kg; from about 1 mg/kg to about 2 mg/kg; from about 1 mg/kg to about 3 mg/kg; from about 1 mg/kg to about 5 mg/kg; from about 2 mg/kg to about 3 mg/kg; from about 1 mg/kg to about 4 mg/kg; or at about 1.2 mg/kg, at about 2.4 mg/kg, or at about 3.6 mg/kg.

In certain embodiments, the positively charged amino acid (e.g., arginine) is present in the pharmaceutical composition at a concentration of greater than about 0.1%; greater than about 0.5%; greater than about 1%; greater than about 5%; greater than about 10%; greater than about 20%; from about 0.5% to about 20%; from 1% to about 10%; from about 5% to about 15%; or at about 5% or at about 10% based on the weight of the pharmaceutical composition.

In certain embodiments, the ratio (w/w) of the positively charged amino acid to the levodopa or pharmaceutically acceptable salt thereof is greater than about 2:1; greater than about 5:1; greater than about 8:1; greater than about 10:1; greater than about 12:1; greater than about 25:1; or greater than about 50:1. In other embodiments, the ratio (w/w) of the positively charged amino acid to the levodopa or pharmaceutically acceptable salt thereof is from about 2:1 to about 50:1; from about 5:1 to about 20:1; from about 8:1 to about 18:1; from about 10:1 to about 15:1; or about 12.5:1.

In certain embodiments, the nasally administered pharmaceutical composition of the invention is concurrently, simultaneously or sequentially administered with orally administered levodopa or a pharmaceutically acceptable salt thereof and a decarboxylase inhibitor, which can be administered orally or by a different route. In particular embodiments, the nasal composition of the present invention is administered to treat “off periods” or breakthrough symptoms associated with an oral levodopa treatment in the patient in need thereof. In alternative embodiments, the nasal administration is the sole levodopa therapy without associated oral levodopa administration.

In certain embodiments, the methods of the invention provide a therapeutically effective amount of levodopa or metabolite thereof (e.g., dopamine) to the brain of the patient.

In certain embodiments, the systemic plasma level of dopamine, attributed by the nasal administration, is minimal, i.e., below detectable limits.

In certain embodiments, the methods of the invention provide a ratio of dopamine maximum concentration in the brain to dopamine maximum concentration in the systemic plasma at a value greater than about 10:1, greater than about 50:1; greater than about 100:1; greater than about 500:1; greater than about 1,000:1; greater than about 5,000:1; or greater than about 10,000:1. In other embodiments, the ratio is from about 10:1 to about 10,000:1; from about 50:1 to about 5,000: or from about 100:1 to about 1,000:1.

In certain embodiments, a dose of the pharmaceutical compositions of the invention may be administered one time a day, two times a day, three times a day or four times a day. In other embodiments, a dose may be administered every 72 hours, every 48 hours, every 24 hours, every 12 hours, every 8 hours, every 6 hours or every 4 hours. In other embodiments, the dose may be administered once weekly, twice weekly, three times weekly, four times weekly or five times weekly. In alternative embodiments, the dose is administered as needed for breakthrough symptoms of oral levodopa therapy.

In certain embodiments, the pharmaceutical compositions of the present invention are administered from a nasal device adapted to deliver the composition to the olfactory region of the nose. In some embodiments, the nasal device can include a suitable container (e.g., made of glass or plastic) to contain the pharmaceutical compositions disclosed herein. The device can be a nasal spray applicator, a squeeze bottle, a dropper bottle with pipette, a rhinyl catheter with a squirt tube, or a metered-dose spray pump. The device can be single use or provide multiple doses. An example of a nasal device that can be used in certain embodiments of the invention is described in U.S. Pat. No. 10,507,295.

In certain embodiments, the invention is directed to a method of delivering levodopa or a pharmaceutically acceptable salt thereof to a patient identified as in need of levodopa therapy, comprising administering to the olfactory region of the nose of the patient a pharmaceutical composition comprising levodopa or pharmaceutically acceptable salt thereof and a positively charged amino acid, wherein said method selectively delivers a therapeutically effective amount of the levodopa or pharmaceutically acceptable salt thereof to the brain tissues of the patient. In these embodiments, the method thereby effectively treats diseases or disorders (such as Parkinson's disease) treatable or manageable by a levodopa therapy in the patient.

### Therapeutically Active Agents

The delivery systems and pharmaceutical compositions disclosed herein include levodopa or a pharmaceutically acceptable salt thereof as a therapeutically active agent. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like. In certain embodiments, the therapeutically effective agent is levodopa free base.

### Positively Charged Amino Acids

The positively charged amino acids of the invention may be lysine, arginine, histidine, and the like, or a combination thereof. In a particular embodiment the positively charged amino acid comprises arginine (i.e., L-arginine) or derivatives thereof. In certain embodiments, the arginine is delivered as arginine glutamate or arginine alpha-ketoglutarate. In certain embodiments, other amino acids that may be incorporated into a composition of the invention include both natural and synthetic amino acid compounds that carry positive charges (e.g., protonated).

### Pharmaceutically Acceptable Excipients

The pharmaceutical compositions according to the invention may comprise one or more pharmaceutically acceptable vehicles, carriers and other excipients appropriate for nasal administration. Examples of possible pharmaceutically acceptable vehicles, carriers and other excipients are described in the Handbook of Pharmaceutical Excipients, American Pharmaceutical Association (6^{th} Edition, 2009 Publication), which is incorporated by reference herein. Vehicles, carriers and other excipients suitable for nasal formulations include, but are not limited to, antioxidants, buffering agents, diluents, surfactants, solubilizers, stabilizers, hydrophilic polymers, additional absorption or permeability enhancers, preservatives, osmotic agents, isotonicity agents, pH adjusting agents, solvents, co-solvents, viscosity agents, gelling agents, suspending agents or combinations thereof.

Suitable surfactants for the formulations disclosed herein include, but are not limited to Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and combinations thereof.

Suitable isotonicity agents for the pharmaceutical compositions disclosed herein include, but are not limited to dextrose, lactose, sodium chloride, calcium chloride, magnesium chloride, sorbitol, sucrose, mannitol, trehalose, raffinose, various polyethylene glycol (PEG), hydroxyethyl starch, glycine, and the like, and combinations thereof.

Suitable suspending agents for the formulations disclosed herein include, but are not limited to microcrystalline cellulose, carboxymethylcellulose sodium NF, polyacrylic acid, magnesium aluminum silicate, xanthan gum, and the like, and mixtures thereof.

Suitable preservatives include phenylethyl alcohol, benzalkonium chloride, benzoic acid, or benzoates such as sodium benzoate.

### EXAMPLES

The following Examples are set forth to assist in understanding the invention and should not be construed as specifically limiting the invention described and claimed herein. Such variations of the invention, including the substitution of any or all equivalents now known or later developed, which would be within the purview of those skilled in the art, and changes in formulation or minor changes in therapeutic design, are to be considered to fall within the scope of the invention incorporated herein.

### Example 1

### Preparation of Formulation

An 8 mg/mL L-Dopa nasal composition was prepared by adding about 0.9 mL of sterile water to a powder mixture of 8 mg of L-Dopa and 100 mg of L-arginine. After measuring pH, acetic acid was used to adjust the solution to pH of 7.5. Finally, water was added until a total volume of 1.0 mL was reached.

### Dosing Solution Analysis

The measured dosing solution concentration is shown in Table 1. The dosing solution was diluted into rat plasma and analyzed in triplicate. All concentrations are expressed as mg/mL. The nominal dosing level was used in all calculations.

TABLE 1
Measured Dosing Solution Concentrations (mg/mL)

| | | | | Measured | |
|---|---|---|---|---|---|
| | | | Nominal | Dosing | |
| | | | Dosing | Solution | |
| | | | Conc. | Conc. | |
| Test | Route of | | (mg/mL | (mg/mL | % of |
| Article | Administration | Vehicle | L-Dopa) | L-Dopa) | Nominal |
| L-Dopa + | IN | Sterile | 8 | 8.74 | 109 |
| Arginine | | Water | | | |

### Quantitative Sample Analysis

Plasma and brain samples were extracted and analyzed using the methods described below.

### Analytical Stock Solution Preparation

Analytical stock solutions (1.00 mg/mL of the free drug) were prepared in DMSO.

### Tissue Homogenization

Brain samples were homogenized with a Virsonic 100 ultrasonic homogenizer. Each brain sample was first weighed, and then 2 mL of 20:80 methanol:water was added to each gram of sample. Samples were then homogenized on ice, and stored frozen until analysis.

### Standard Preparation

Standards were prepared in Sprague-Dawley rat plasma containing K₂EDTA as the anticoagulant or rat brain homogenate. Plasma samples were treated 10:1 with 25% sodium metabisulfide. Working solutions were prepared in 50:50 acetonitrile:water. Working solutions were then added to the matrix to make calibration standards to final concentrations of 1000, 500, 250, 100, 50.0, 20.0, 10.0, and 5.00 ng/mL. Standards were treated identically to the study samples.

### Sample Extraction

Plasma and brain samples were manually extracted in snap cap microcentrifuge tubes.

| Steps | Procedure |
|---|---|
| 1 | Standards: Add 10 μL of appropriate working solution |
| | to 50 mL of blank matrix. |
| | Blanks: Add 20 μL of water to 50 mL blank matrix |
| | for double blanks. Add 10 μL of water for blanks with IS |
| | Samples: Add 10 μL water to 50 mL of study sample and blanks. |
| | Add 10 μL ISWS to all samples except double blank matrix. |
| 2 | Add 50 μL of ice cold 2N perchloric acid containing 5 μg/mL |
| | L-Dopa-d₃ as internal standard. Cap and vortex well. |
| 3 | Centrifuge samples at 13,000 rpm for 10 minutes. |
| 4 | Transfer supernatant to a clean 96-well plate and inject. |

### HPLC Conditions

### Instrument: Waters Acquity UPLC

### Column: Waters Acquity BEH C18, 100×2.1 mm id, 1.7 μm

Aqueous Reservoir (A): 0.1% formic acid in water
Organic Reservoir (B): 100% methanol Gradient Program:

| Time (min) | Grad. Curve | % A | % B |
|---|---|---|---|
| 0.00 | 6 | 99 | 1 |
| 1.00 | 6 | 99 | 1 |
| 2.00 | 6 | 5 | 95 |
| 3.00 | 6 | 5 | 95 |
| 3.10 | 6 | 99 | 1 |
| 4.00 | 6 | 99 | 1 |

### Flow Rate: 400 μL/min

### Injection Volume: 10 μL

### Run Time: 4.00 min

### Column Temperature: 40° C.

### Sample Temperature: 8° C.

Strong Autosampler Wash: 1:1:1 (v:v:v) H₂O:MeCN:IPA with 0.2% formic acid
Weak Autosampler Wash: 0.1% formic acid in water

### Mass Spectrometer Conditions

### Instrument: Waters Xevo TQ-S MS

### Interface: Electrospray

### Mode: Multiple Reactions Monitoring (MRM)

Collision Gas: 0.41 mL/min

### Desolvation Temp: 600° C.

### Capillary Voltage: 2.8 kV

### Nebulizer Gas Flow: 7 Bar

| | | | | Collision | Cone |
|---|---|---|---|---|---|
| | | Precursor | Product | Energy | Voltage |
| Analyte | Polarity | Ion | Ion | (eV) | (V) |
| L-Dopa | Positive | 198.1 | 152.1 | 14 | 24 |
| Dopamine | Positive | 154.1 | 91.1 | 14 | 24 |
| L-Dopa-d₃ | Positive | 200.1 | 154.1 | 14 | 24 |
| (Internal Standard) | | | | | |

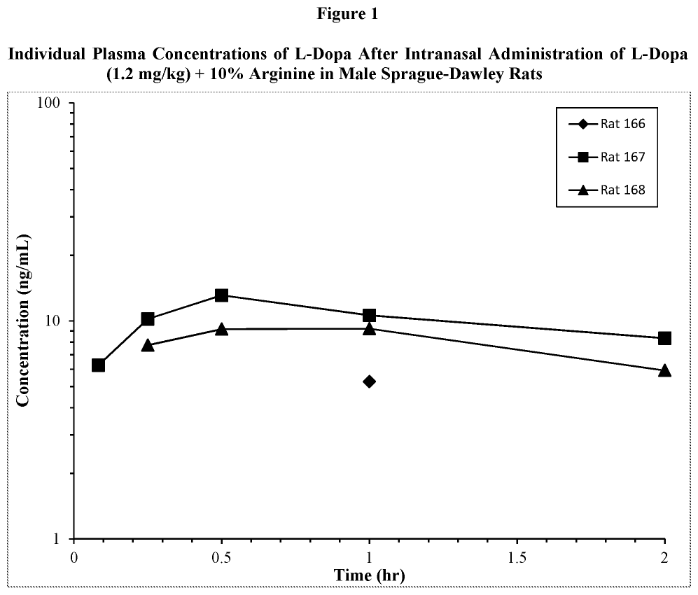

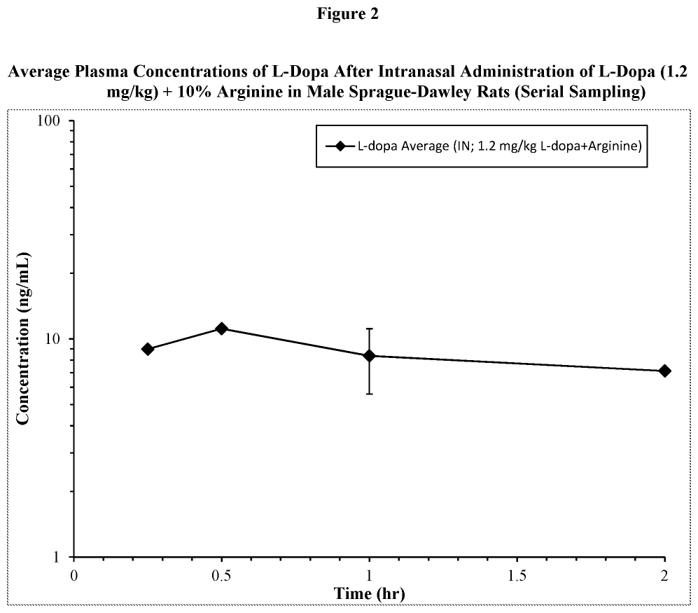

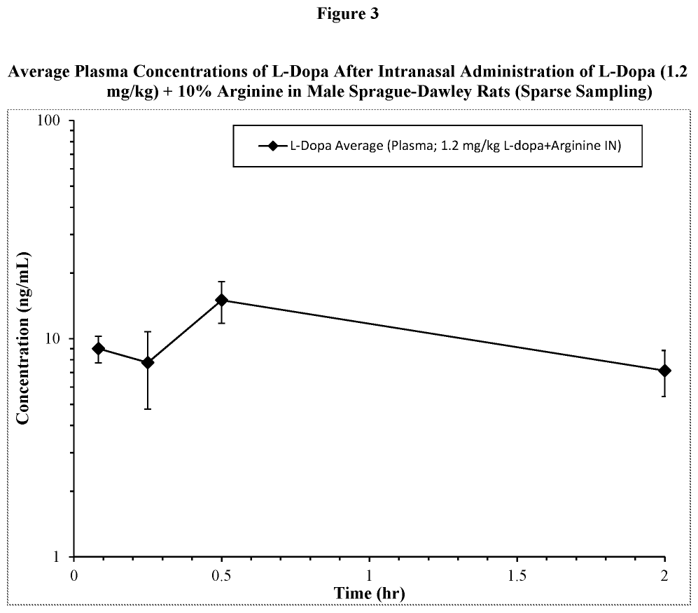

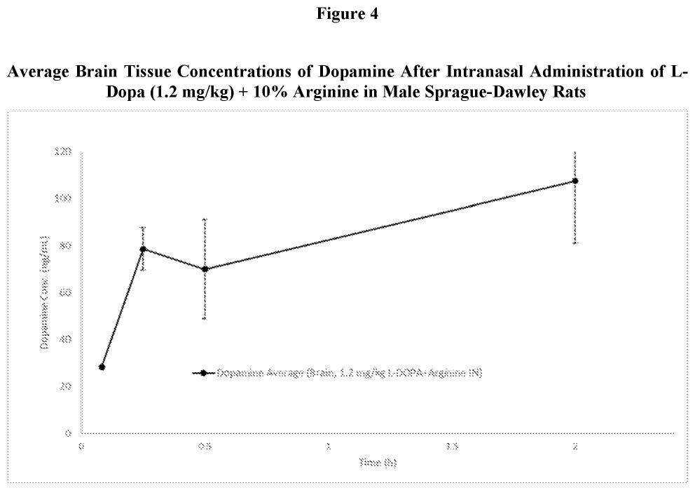

Individual and average plasma concentrations of L-Dopa and dopamine are shown in Tables 2, 3 and 4. The data are expressed as ng/mL of either L-Dopa or dopamine. Brain tissue concentrations are shown in Table 5. The data are expressed as ng of L-Dopa or dopamine per gram tissue. Samples that were below the limit of quantification were not used in the calculation of averages. Plasma concentrations versus time data are plotted in FIG. 1 through FIG. 3. Brain tissue concentrations versus time data are plotted in FIG. 4.

### Data Analysis

Pharmacokinetic parameters were calculated from the time course of the plasma and brain tissue concentrations and are presented in Tables 2, 3 and 4. Pharmacokinetic parameters were determined with Phoenix WinNonlin (v8.0) software using a non-compartmental model with or without sparse sampling. The sparse sampling option uses the mean concentration of the triplicate samples at each time point. This was used because of the limited number of samples per subject. The maximum plasma concentration (Cₘₐₓ) and the time to reach maximum plasma concentration (tₘₐₓ) after IN dosing were observed from the data. The area under the time-concentration curve (AUC) was calculated using the linear trapezoidal rule with calculation to the last quantifiable data point, and with extrapolation to infinity if applicable. Plasma and brain half-life (t_{1/2}) were calculated from 0.693/slope of the terminal elimination phase. Mean residence time (MRT) was calculated by dividing the area under the moment curve (AUMC) by the AUC. Any samples below the limit of quantitation were treated as zero for the pharmacokinetic data analysis.

TABLE 2
Individual and Average Plasma Concentrations (ng/mL)
and Pharmacokinetic Parameters for L-Dopa After
Intranasal Administration of L-Dopa (1.2 mg/kg) + 10%
Arginine in Male Sprague-Dawley Rats
Intranasal (L-Dopa; dosed 1.2 mg/kg L-Dopa + Arginine)

| | Rat # | | | | |
|---|---|---|---|---|---|
| Time (hr) | 166 | 167 | 168 | Mean | SD |
| 0 (pre-dose) | BLOQ | BLOQ | BLOQ | ND | ND |
| 0.083 | BLOQ | 6.26 | BLOQ | ND | ND |
| 0.25 | BLOQ | 10.2 | 7.73 | 8.97 | ND |
| 0.50 | BLOQ | 13.1 | 9.16 | 11.1 | ND |
| 1.0 | 5.26 | 10.6 | 9.21 | 8.36 | 2.77 |
| 2.0 | BLOQ | 8.33 | 5.92 | 7.13 | ND |
| Animal Weight (kg) | 0.312 | 0.308 | 0.313 | 0.311 | 0.003 |
| Volume Dosed (mL) | 0.047 | 0.046 | 0.047 | 0.047 | 0.001 |
| Cₘₐₓ (ng/mL) | 5.26 | 13.1 | 9.21 | 9.19 | 3.92 |
| tₘₐₓ (hr) | 1.0 | 0.50 | 1.0 | 0.83 | 0.29 |
| t_{1/2} (hr) | ND | ND² | ND² | ND | ND |
| MRTₗₐₛₜ (hr) | ND | 0.970 | 1.00 | 0.986 | ND |
| AUCₗₐₛₜ (hr · ng/mL) | ND | 19.9 | 14.9 | 17.4 | ND |
| AUC_{∞} (hr · ng/mL) | ND | ND² | ND² | ND | ND |
| Dose-normalized Values¹ | | | | | |
| AUCₗₐₛₜ | ND | 16.6 | 12.4 | 14.5 | ND |
| (hr · kg · ng/mL/mg) | | | | | |
| AUC_{∞} | ND | ND² | ND² | ND | ND |
| (hr · kg · ng /mL/mg) | | | | | |

Cₘₐₓ: maximum plasma concentration; tₘₐₓ: time of maximum plasma concentration; t_{1/2}: half-life, data points used for half-life determination are in bold; MRTₗₐₛₜ: mean residence time, calculated to the last observable time point; AUCₗₐₛₜ: area under the curve, calculated to the last observable time point; AUC_{∞}: area under the curve, extrapolated to infinity; ND: not determined; BLOQ: below the limit of quantitation (5.00 ng/mL).
¹Dose-normalized by dividing the parameter by the nominal dose in mg/kg.
²Not determined due to a lack of quantifiable data points trailing the Cₘₐₓ.

TABLE 3
Plasma Concentrations (ng/mL) and Pharmacokinetic Parameters
for L-Dopa After Intranasal Administration of L-Dopa
(1.2 mg/kg) + 10% Arginine in Male Sprague-Dawley Rats
Intranasal (L-Dopa; dosed 1.2 mg/kg L-Dopa + Arginine)

| Time (hr) | Rat # | Conc. (ng/mL) | Mean (ng/mL) | SD (ng/mL) |
|---|---|---|---|---|
| 0 (pre-dose) | 156 | BLOQ | NA | NA |
| 0.083 | 157 | 7.56 | 9.00 | 1.26 |
| | 158 | 9.89 | | |
| | 159 | 9.6 | | |
| 0.25 | 160 | 11 | 7.76 | 3.01 |
| | 161 | 7.24 | | |
| | 162 | 5.05 | | |
| 0.50 | 163 | 17.3 | 15.0 | 3.25 |
| | 164 | 63.0* | | |
| | 165 | 12.7 | | |
| 2.0 | 166 | BLOQ | 7.13 | 1.70 |
| | 167 | 8.33 | | |
| | 168 | 5.92 | | |

| | |
|---|---|
| Cₘₐₓ (ng/mL) ± SE | 15.0 ± 2.30 |
| tₘₐₓ (hr) | 0.50 |
| t_{1/2} (hr) | ND |
| MRTₗₐₛₜ (hr) | 0.634 |
| AUCₗₐₛₜ (hr · ng/mL) ± SE | 19.4 ± 2.76 |
| AUC_{∞} (hr · ng/mL) | ND |
| Dose-normalized Values¹ | |
| AUCₗₐₛₜ (hr · kg · ng/mL/mg) ± SE | 16.2 ± 2.30 |
| AUC_{∞} (hr · kg · ng/mL/mg) | ND |

Cₘₐₓ: maximum plasma concentration; tₘₐₓ: time of maximum plasma concentration; t_{1/2}: half-life; time points used are in bold text; MRTₗₐₛₜ: mean residence time, calculated to the last observable time point; AUCₗₐₛₜ: area under the curve, calculated to the last observable time point; AUC_{∞}: area under the curve, extrapolated to infinity. ND: not determined; NA: not applicable; BLOQ: below the limit of quantitation (5.00 ng/mL).
¹Dose-normalized by dividing the parameter by the nominal dose in mg/kg.
*Excluded from all pharmacokinetic parameter calculations and averages due to being an outlier.

TABLE 4
Individual and Average Plasma Concentrations (ng/mL)
and Pharmacokinetic Parameters for Dopamine After
Intranasal Administration of L-Dopa (1.2 mg/kg) + 10%
Arginine in Male Sprague-Dawley Rats
Intranasal Dopamine; dosed 1.2 mg/kg L-Dopa + Arginine)

| | Rat # | | | | |
|---|---|---|---|---|---|
| Time (hr) | 166 | 167 | 168 | Mean | SD |
| 0 (pre-dose) | BLOQ | BLOQ | BLOQ | ND | ND |
| 0.083 | BLOQ | BLOQ | BLOQ | ND | ND |
| 0.25 | BLOQ | BLOQ | BLOQ | ND | ND |
| 0.50 | BLOQ | BLOQ | BLOQ | ND | ND |
| 1.0 | BLOQ | BLOQ | BLOQ | ND | ND |
| 2.0 | BLOQ | BLOQ | BLOQ | ND | ND |

ND: not determined; BLOQ: below the limit of quantitation (5.00 ng/mL).

TABLE 5
Individual and Average Brain Tissue Concentrations for Dopamine
After Intranasal Administration of L-Dopa (1.2 mg/kg) + 10%
Arginine in Male Sprague-Dawley Rats
Brain Tissue (Dopamine; dosed 1.2 mg/kg L-Dopa + Arginine)

| | | | | Brain | Brain |
|---|---|---|---|---|---|
| | | | Brain | Homogenate | Homogenate |
| | Time | Rat | Weight | Volume | Conc. |
| Analyte | (hr) | # | (g) | (mL) | (ng/mL) |
| Dopamine | 0.083 | 157 | 1.87 | 5.61 | 31.4 |
| | 0.083 | 158 | 1.86 | 5.58 | 18.0 |
| | 0.083 | 159 | 1.91 | 5.73 | 35.4 |
| | 0.25 | 160 | 1.87 | 5.61 | 65.5 |
| | 0.25 | 161 | 1.87 | 5.61 | 67.3 |
| | 0.25 | 162 | 1.93 | 5.79 | 103 |
| | 0.50 | 163 | 1.85 | 5.55 | 77.3 |
| | 0.50 | 164 | 1.77 | 5.31 | 40.8 |
| | 0.50 | 165 | 1.91 | 5.73 | 92.0 |
| | 2.0 | 166 | 1.86 | 5.58 | 101 |
| | 2.0 | 167 | 2.01 | 6.03 | 114 |
| | 2.0 | 168 | 1.91 | 5.73 | 102 |

### Summary of Results

In this study, the exposure of levodopa (L-Dopa) and its metabolite, dopamine, was evaluated following intranasal (IN) administration of a formulation (L-Dopa+arginine) in male Sprague-Dawley rats. Blood and brain tissue samples were collected up to 2 hours post-dose, and plasma and brain concentrations of L-Dopa and dopamine (some of which may be endogenous) were determined by LC-MS/MS. Pharmacokinetic parameters were determined using Phoenix WinNonlin (v8.0) software with or without sparse sampling.

Following IN dosing of L-Dopa (at 1.2 mg/kg)+10% Arginine, maximum plasma concentrations (average of 9.19±3.92 ng/mL) of L-Dopa following serial sampling were observed between 30 minutes and 1 hour post dosing. The average total exposure (AUCₗₐₛₜ) of L-Dopa following serial sampling was 17.4 hr*ng/mL and the dose-normalized AUCₗₐₛₜ was 14.5 hr*kg*ng/mL/mg. Following IN dosing of L-Dopa (at 1.2 mg/kg)+10% Arginine, the average (±SE) Cₘₐₓ of L-Dopa in plasma following sparse sampling was 15.0±2.30 ng/mL, the tₘₐₓ was 30 minutes, the half-life could not be determined, the exposure based on the average (±SE) dose normalized AUCₗₐₛₜ was 16.2±2.30 hr*kg*ng/mL/mg. All L-Dopa concentrations in brain tissue were below the limit of quantitation.

Following IN dosing of L-Dopa (at 1.2 mg/kg)+10% Arginine, the average (±SE) Cₘₐₓ of dopamine in brain tissue was 105.7±7.2 ng/mL. All dopamine concentrations in plasma were below the limit of quantitation.

### Example 2

### Preparation of Formulation

Three different dosing concentrations were tested: Group (1), L-Dopa (2.4 mg/kg)+10% Arginine; Group (2), L-Dopa (3.6 mg/kg)+10% Arginine; and Group (3), L-Dopa (2.4 mg/kg)+5% Arginine. To prepare the dose for Group (1), a 16 mg/mL L-Dopa nasal composition was prepared by adding about 0.9 mL of sterile water to a powder mixture of 16 mg of L-Dopa and 100 mg of L-arginine. After measuring pH, acetic acid was used to adjust the solution to pH of 7.5. Finally, water was added until a total volume of 1.0 mL was reached. To prepare the dose for Group (2), similar steps were used as described for Group (1). That is, a 24 mg/mL L-Dopa nasal composition was prepared by adding about 0.9 mL of sterile water to a powder mixture of 24 mg of L-Dopa and 100 mg of L-arginine. After measuring pH, acetic acid was used to adjust the solution to pH of 7.5. Finally, water was added until a total volume of 1.0 mL was reached. The dose for Group (3) was prepared as follows, a 16 mg/mL L-Dopa nasal composition was prepared by adding about 0.9 mL of sterile water to a powder mixture of 16 mg of L-Dopa and 50 mg of L-arginine. After measuring pH, acetic acid was used to adjust the solution to pH of 7.5. Finally, water was added until a total volume of 1.0 mL was reached.

### Study Design

The study design using the dosing prepared for Groups (1), (2), and (3) is shown in Table 6.

TABLE 6
Study Design

| | | | | | Dosing | | | | | |
|---|---|---|---|---|---|---|---|---|---|---|
| | | | | | Solution | | | | | |
| | | | Total | Dose | Conc. | Dosing | | Blood Sampling | Brain | |
| Group | | Dosing | Animals | (mg/kg L- | (mg/mL | Volume | | Time | Collection Time | |
| # | Test Article | Route | N= | Dopa) | L-Dopa) | (mL/kg) | Vehicle | Points (n = 5) | Points* | Analytes |
| 1 | L-Dopa + | IN | 40 | 2.4 mg/kg | 16 | 0.15 | water | Pre-dose(0), | Pre-dose(0)^{#}, | L-Dopa, |
| | L-Arginine | | | L-Dopa + | | | | 5, 15, 30 min | 5, 15, 30 min | dopamine |
| | (A) | | | 10% ARG | | | | 1, 2, 4, and 8* | 1, 2, 4, and 8* | |
| | | | | | | | | hrs | hrs | |
| 2 | L-Dopa + | IN | 35 | 3.6 mg/kg | 24 | 0.15 | water | Pre-dose(0), | Pre-dose(0), | L-Dopa, |
| | L-Arginine | | | L-Dopa + | | | | 5, 15, 30 min | 5, 15, 30 min | dopamine |
| | (B) | | | 10% ARG | | | | 1, 2, 4, and 8 | 1, 2, 4, and 8* | |
| | | | | | | | | hrs | hrs | |
| 3 | L-Dopa + | IN | 35 | 2.4 mg/kg | 16 | 0.15 | water | Pre-dose(0), | Pre-dose(0), | L-Dopa, |
| | L-Arginine | | | L-Dopa + | | | | 5, 15, 30 min | 5, 15, 30 min | dopamine |
| | (C) | | | 5% ARG | | | | 1, 2, 4, and 8 | 1, 2, 4, and 8* | |
| | | | | | | | | hrs | hrs | |

*Rats being euthanized at 8 hrs for brain collection, will have blood collections at all 8 time points, from pre-dose to 8 hrs. For all other brain collection time points, a discrete group of n-5 rats will be dosed and sacrificed at each time point (no blood collections will be performed for these animals).
^{#}Pre-dose animals from Group 1 (for brain collections) will be shared across all dose groups.

### Analytical Stock Solution Preparation

Analytical stock solutions (1.00 mg/mL of the free drug) were prepared in DMSO.

### Tissue Homogenization

Brain samples were homogenized with a Virsonic 100 ultrasonic homogenizer. Each brain sample was first weighed, and then 2 mL of 20:80 methanol:water was added to each gram of sample. Samples were then homogenized on ice, and stored frozen until analysis.

### Standard Preparation

Standards were prepared in Sprague-Dawley rat plasma containing K₂EDTA as the anticoagulant or rat brain homogenate. Plasma samples were treated 10:1 with 25% sodium metabisulfide. Working solutions were prepared in 50:50 acetonitrile:water. Working solutions were then added to the matrix to make calibration standards to final concentrations of 1000, 500, 250, 100, 50.0, 20.0, 10.0, and 5.00 ng/mL. Standards were treated identically to the study samples.

### Sample Extraction

Plasma and brain samples were manually extracted in snap cap microcentrifuge tubes. The samples were analyzed as described in Example 1.

The results are presented in the following Tables.

TABLE 7
Individual and Mean Average Plasma Concentrations (ng/mL) and
Pharmacokinetic Parameters for L-Dopa After Intranasal Administration of L-Dopa
(2.4 mg/kg) + 10% Arginine Male Sprague-Dawley Rats (Group (1))

| | Intranasal (L-Dopa; dosed 2.4 mg/kg L-Dopa + 10% Arginine) | | | | | | | |
|---|---|---|---|---|---|---|---|---|
| | Sparse | Rat # | | | | | | |
| Time (hr) | Mean ± SE | 680 | 681 | 682 | 683 | 684 | Mean | SD |
| 0 (pre-dose) | BLOQ | BLOQ | BLOQ | BLOQ | BLOQ | BLOQ | BLOQ | NA |
| 0.083 | 8.96 | 5.63 | 21.7 | 9.21 | BLOQ | 5.62 | 10.2 | 6.6 |
| 0.25 | 39.7 ± 43.4 | 15.6 | 20.4 | 16.5 | 5.3 | 16.3 | 19.0 | 11.3 |
| 0.50 | 21.6 ± 4.9 | 22.2 | 11.5 | 21.0 | 7.84 | 24.2 | 18.1 | 6.7 |
| 1.0 | 47.3 ± 26.0 | 18.4 | 11.6 | 21.2 | 5.90 | 18.6 | 20.5 | 14.3 |
| 2.0 | 26.8 ± 19.5 | 16.7 | 12.9 | 9.33 | BLOQ | 16.8 | 16.5 | 6.5 |
| 4.0 | 7.44 | BLOQ | BLOQ | BLOQ | BLOQ | BLOQ | BLOQ | NA |
| 8.0 | BLOQ | BLOQ | BLOQ | BLOQ | BLOQ | BLOQ | BLOQ | NA |
| t_{1/2} (hr) | ND² | ND³ | ND² | ND² | ND² | ND² | NA | NA |
| tₘₐₓ (hr) | 1.0 | 0.5 | 0.083 | 1.0 | 0.5 | 0.5 | 0.597 | 0.351 |
| Cₘₐₓ (ng/mL) | 47.3 ± 11.6 | 22.2 | 21.7 | 21.2 | 7.84 | 24.2 | 24.1 | 12.8 |
| MRTₗₐₛₜ (hr) | 1.42 | 1.03 | 0.948 | 0.912 | 0.584 | 1.02 | 0.986 | 0.269 |
| AUCₗₐₛₜ | 96.2 ± 16.4 | 34.4 | 26.4 | 33.0 | 5.52 | 35.5 | 38.5 | 30.4 |
| (hr · ng/mL) | | | | | | | | |
| AUC_{∞} | ND² | ND³ | ND² | ND² | ND² | ND² | NA | NA |
| (hr · ng/mL) | | | | | | | | |
| Dose- | | | | | | | | |
| normalized | | | | | | | | |
| Values¹ | | | | | | | | |
| AUCₗₐₛₜ | 40.1 | 14.3 | 11.0 | 13.8 | 2.30 | 14.8 | 16.1 | 12.7 |
| (hr · kg · ng/ | | | | | | | | |
| mL/mg) | | | | | | | | |
| AUC_{∞} | ND² | ND³ | ND² | ND² | ND² | ND² | NA | NA |
| (hr · kg · ng/ | | | | | | | | |
| mL/mg) | | | | | | | | |

Cₘₐₓ: maximum plasma concentration;
tₘₐₓ: time of maximum plasma concentration;
t_{1/2}: half-life, data points used for half-life determination are in bold;
MRTₗₐₛₜ: mean residence time, calculated to the last observable time point;
AUCₗₐₛₜ: area under the curve, calculated to the last observable time point;
AUC_{∞}: area under the curve, extrapolated to infinity;
ND: not determined;
BLOQ: below the limit of quantitation (5.00 ng/mL).
¹Dose-normalized by dividing the parameter by the nominal dose in mg/kg.
²Not determined due to a lack of quantifiable data points trailing the Cₘₐₓ.

TABLE 8
Individual and Mean Concentrations (ng/mL) of Dopamine in Brain
Tissue After Intranasal Administration of L-Dopa (2.4 mg/kg) + 10%
Arginine in Male Sprague-Dawley Rats (Group 1)

| Time (hr) | Sparse Mean ± SD |
|---|---|
| 0 | 2.86 ± 6.4 |
| 0.083 | 18.5 ± 4.6 |
| 0.25 | 12.5 ± 7.3 |
| 0.50 | 12.7 ± 7.6 |
| 1.0 | 18.0 ± 4.0 |
| 2.0 | 3.33 ± 7.4 |
| 4.0 | 0 |
| 8.0 | 6.0 ± 8.5 |

PK Parameters

| | |
|---|---|
| t_{1/2} (hr) | ND² |
| tₘₐₓ (hr) | 0.083 |
| Cₘₐₓ (ng/mL) | 18.5 ± 2.0 |
| MRTₗₐₛₜ (hr) | 3.05 |
| AUCₗₐₛₜ | 40.3 ± 9.3 |
| (hr · ng/mL) | |
| AUC_{∞} | ND² |
| (hr · ng/mL) | |

Dose Normalized

| | |
|---|---|
| AUCₗₐₛₜ | 16.8 |
| (hr · kg · ng/mL/mg) | |
| AUC_{∞} | ND² |
| (hr · kg · ng/mL/mg) | |

Cₘₐₓ: maximum plasma concentration; tₘₐₓ: time of maximum plasma concentration; t_{1/2}: half-life; time points used are in bold text; MRTₗₐₛₜ: mean residence time, calculated to the last observable time point; AUCₗₐₛₜ: area under the curve, calculated to the last observable time point; AUC_{∞}: area under the curve, extrapolated to infinity. ND: not determined; NA: not applicable; BLOQ: below the limit of quantitation (5.00 ng/mL).
¹Dose-normalized by dividing the parameter by the nominal dose in mg/kg.
*Excluded from all pharmacokinetic parameter calculations and averages due to being an outlier.

TABLE 9
Individual and Mean Average Plasma Concentrations (ng/mL) and
Pharmacokinetic Parameters for L-Dopa After Intranasal Administration of L-Dopa
(3.6 mg/kg) + 10% Arginine in Male Sprague-Dawley Rats (Group (2))

| | Intranasal (L-Dopa; dosed 3.6 mg/kg L-Dopa + 10% Arginine) | | | | | | | |
|---|---|---|---|---|---|---|---|---|
| | Sparse | Rat # | | | | | | |
| Time (hr) | Mean ± SE | 715 | 716 | 717 | 718 | 719 | Mean | SD |
| 0 (pre-dose) | BLOQ | BLOQ | BLOQ | BLOQ | BLOQ | BLOQ | BLOQ | NA |
| 0.083 | 7.87 ± 6.21 | BLOQ | BLOQ | BLOQ | BLOQ | 6.53 | 7.20 | NA |
| 0.25 | 24.2 ± 13.1 | 7.16 | 8.80 | 10.2 | 15.1 | 12.7 | 13.0 | 6.2 |
| 0.50 | 18.5 ± 8.69 | 7.56 | 12.6 | 14.3 | 24.4 | 16.2 | 15.6 | 5.7 |
| 1.0 | 40.0 ± 35.4 | 8.43 | 13.6 | 12.8 | 23.6 | 10.3 | 18.1 | 11.9 |
| 2.0 | 23.9 ± 10.8 | 6.31 | 13.1 | 12.9 | 39.9 | 13.8 | 18.3 | 12.0 |
| 4.0 | 7.38 | BLOQ | 6.70 | BLOQ | 6.25 | BLOQ | 6.78 | 0.57 |
| 8.0 | BLOQ | 77.1 | BLOQ | BLOQ | BLOQ | 52.3 | 64.7 | NA |
| t_{1/2} (hr) | ND² | ND² | ND² | ND² | ND² | ND² | NA | NA |
| tₘₐₓ (hr) | 1.0 | 8.0 | 1.0 | 0.5 | 2.0 | 8.0 | 3.42 | 3.58 |
| Cₘₐₓ (ng/mL) | 40.0 ± 17.7 | 77.1 | 13.6 | 14.3 | 39.9 | 52.3 | 35.9 | 24.0 |
| MRTₗₐₛₜ (hr) | 4.00 | 7.23 | 1.84 | 1.09 | 1.75 | 6.24 | 3.69 | 2.57 |
| AUCₗₐₛₜ | 141 ± 36.6 | 174 | 43.1 | 23.5 | 96.1 | 143 | 103 | 60.1 |
| (hr · ng/mL) | | | | | | | | |
| AUC_{∞} | ND² | ND² | ND² | ND² | ND² | ND² | NA | NA |
| (hr · ng/mL) | | | | | | | | |
| Dose- | | | | | | | | |
| normalized | | | | | | | | |
| Values¹ | | | | | | | | |
| AUCₗₐₛₜ | 39.3 | 48.4 | 12.0 | 6.54 | 26.7 | 39.6 | 28.8 | 16.7 |
| (hr · kg · ng/ | | | | | | | | |
| mL/mg) | | | | | | | | |
| AUC_{∞} | ND² | ND³ | ND² | ND² | ND² | ND² | NA | NA |
| (hr · kg · ng/ | | | | | | | | |
| mL/mg) | | | | | | | | |

Cₘₐₓ: maximum plasma concentration;
tₘₐₓ: time of maximum plasma concentration;
t_{1/2}: half-life, data points used for half-life determination are in bold;
MRTₗₐₛₜ: mean residence time, calculated to the last observable time point;
AUCₗₐₛₜ: area under the curve, calculated to the last observable time point;
AUC_{∞}: area under the curve, extrapolated to infinity;
ND: not determined;
BLOQ: below the limit of quantitation (5.00 ng/mL).
¹Dose-normalized by dividing the parameter by the nominal dose in mg/kg.
²Not determined due to a lack of quantifiable data points trailing the Cₘₐₓ.

TABLE 10
Individual and Mean Concentrations (ng/mL) of Dopamine
in Brain Tissue After Intranasal Administration
of L-Dopa (3.6 mg/kg) + 10% Arginine in
Male Sprague-Dawley Rats (Group (2))

| Time (hr) | Sparse Mean ± SD |
|---|---|
| 0 | 14.8 ± 9.2 |
| 0.083 | 82.9 ± 34.2 |
| 0.25 | 61.4 ± 42.6 |
| 0.50 | 97.9 ± 17.2 |
| 1.0 | 155 ± 53.8 |
| 2.0 | 143 ± 83.7 |
| 4.0 | 93.2 ± 49.2 |
| 8.0 | 62.8 ± 75.0 |

PK Parameters

| | |
|---|---|
| t_{1/2} (hr) | 5.26 |
| tₘₐₓ (hr) | 1.0 |
| Cₘₐₓ (ng/mL) | 155 ± 24.1 |
| MRTₗₐₛₜ (hr) | 3.38 |
| AUCₗₐₛₜ | 796 ± 116 |
| (hr · ng/mL) | |
| AUC_{∞} | ND² |
| (hr · ng/mL) | |

Dose Normalized

| | |
|---|---|
| AUCₗₐₛₜ | 221 |
| (hr · kg · ng/mL/mg) | |
| AUC_{∞} | ND² |
| (hr · kg · ng/mL/mg) | |

Cₘₐₓ: maximum plasma concentration; tₘₐₓ: time of maximum plasma concentration; t_{1/2}: half-life; time points used are in bold text; MRTₗₐₛₜ: mean residence time, calculated to the last observable time point; AUCₗₐₛₜ: area under the curve, calculated to the last observable time point; AUC_{∞}: area under the curve, extrapolated to infinity. ND: not determined; NA: not applicable; BLOQ: below the limit of quantitation (5.00 ng/mL).
¹Dose-normalized by dividing the parameter by the nominal dose in mg/kg.
*Excluded from all pharmacokinetic parameter calculations and averages due to being an outlier.

TABLE 11
Individual and Mean Average Plasma Concentrations (ng/mL) and
Pharmacokinetic Parameters for L-Dopa After Intranasal Administration of L-Dopa
(2.4 mg/kg) + 5% Arginine in Male Sprague-Dawley Rats (Group (3))

| | Intranasal (L-Dopa; dosed 2.4 mg/kg L-Dopa + 5% Arginine) | | | | | | | |
|---|---|---|---|---|---|---|---|---|
| | Sparse | Rat # | | | | | | |
| Time (hr) | Mean ± SE | 750 | 751 | 752 | 753 | 754 | Mean | SD |
| 0 (pre-dose) | BLOQ | BLOQ | BLOQ | BLOQ | BLOQ | BLOQ | BLOQ | NA |
| 0.083 | BLOQ | BLOQ | BLOQ | BLOQ | BLOQ | BLOQ | BLOQ | NA |
| 0.25 | 16.1 ± 11.3 | BLOQ | 14.7 | 12.4 | 12.0 | 10.8 | 13.2 | 2.2 |
| 0.50 | 18.2 ± 8.40 | BLOQ | 21.0 | 14.2 | 18.0 | 14.2 | 17.1 | 2.9 |
| 1.0 | 17.3 ± 7.1 | 5.02 | 24.9 | 17.8 | 22.0 | 38.4 | 20.9 | 10.9 |
| 2.0 | 12.2 ± 4.3 | 6.59 | 20.9 | 14.9 | 28.0 | 55.1 | 22.9 | 17.4 |
| 4.0 | 10.4 ± 5.02 | BLOQ | BLOQ | BLOQ | BLOQ | 13.5 | 12.0 | NA |
| 8.0 | BLOQ | BLOQ | BLOQ | BLOQ | BLOQ | BLOQ | BLOQ | NA |
| t_{1/2} (hr) | 2.02 | ND² | ND² | ND² | ND² | ND² | NA | NA |
| tₘₐₓ (hr) | 0.5 | 2.0 | 1.0 | 1.0 | 2.0 | 2.0 | 1.42 | 0.66 |
| Cₘₐₓ (ng/mL) | 18.2 ± 3.8 | 6.59 | 24.9 | 17.8 | 28 | 55.1 | 25.1 | 16.4 |
| MRTₗₐₛₜ (hr) | 1.65 | 1.47 | 1.11 | 1.10 | 1.22 | 1.90 | 1.41 | 0.32 |
| AUCₗₐₛₜ | 47.7 ± 5.1 | 7.06 | 40.1 | 28.7 | 39.8 | 133 | 49.3 | 43.2 |
| (hr · ng/mL) | | | | | | | | |
| AUC_{∞} | ND³ | ND² | ND² | ND² | ND² | ND² | NA | NA |
| (hr · ng/mL) | | | | | | | | |
| Dose- | | | | | | | | |
| normalized | | | | | | | | |
| Values¹ | | | | | | | | |
| AUCₗₐₛₜ | 19.9 | 2.94 | 16.7 | 12.0 | 16.6 | 55.2 | 20.5 | 18.0 |
| (hr · kg · ng/ | | | | | | | | |
| mL/mg) | | | | | | | | |
| AUC_{∞} | ND³ | ND² | ND² | ND² | ND² | ND² | NA | NA |
| (hr · kg · ng/ | | | | | | | | |
| mL/mg) | | | | | | | | |

Cₘₐₓ: maximum plasma concentration;
tₘₐₓ: time of maximum plasma concentration;
t_{1/2}: half-life, data points used for half-life determination are in bold;
MRTₗₐₛₜ: mean residence time, calculated to the last observable time point;
AUCₗₐₛₜ: area under the curve, calculated to the last observable time point;
AUC_{∞}: area under the curve, extrapolated to infinity;
ND: not determined;
BLOQ: below the limit of quantitation (5.00 ng/mL).
¹Dose-normalized by dividing the parameter by the nominal dose in mg/kg.
²Not determined due to a lack of quantifiable data points trailing the Cₘₐₓ.

TABLE 12
Individual and Mean Concentrations (ng/mL) of Dopamine
in Brain Tissue After Intranasal Administration
of L-Dopa (2.4 mg/kg) + 5% Arginine in
Male Sprague-Dawley Rats (Group (3))

| Time (hr) | Sparse Mean ± SD |
|---|---|
| 0 | 14.8 ± 9.2 |
| 0.083 | 6.24 ± 14.0 |
| 0.25 | 5.46 ± 12.2 |
| 0.50 | 25.9 ± 26.0 |
| 1.0 | 50.3 ± 60.4 |
| 2.0 | 1.08 ± 2.41 |
| 4.0 | 0 |
| 8.0 | 11.8 ± 26.4 |

PK Parameters

| | |
|---|---|
| t_{1/2} (hr) | ND² |
| tₘₐₓ (hr) | 1.0 |
| Cₘₐₓ (ng/mL) | 50.3 ± 27.0 |
| MRTₗₐₛₜ (hr) | 3.13 |
| AUCₗₐₛₜ | 75.2 ± 31.5 |
| (hr · ng/mL) | |
| AUC_{∞} | ND² |
| (hr · ng/mL) | |

Dose Normalized

| | |
|---|---|
| AUCₗₐₛₜ | 31.3 |
| (hr · kg · ng/mL/mg) | |
| AUC_{∞} | ND² |
| (hr · kg · ng/mL/mg) | |

Cₘₐₓ: maximum plasma concentration; tₘₐₓ: time of maximum plasma concentration; t_{1/2}: half-life; time points used are in bold text; MRTₗₐₛₜ: mean residence time, calculated to the last observable time point; AUCₗₐₛₜ: area under the curve, calculated to the last observable time point; AUC_{∞}: area under the curve, extrapolated to infinity. ND: not determined; NA: not applicable; BLOQ: below the limit of quantitation (5.00 ng/mL).
¹Dose-normalized by dividing the parameter by the nominal dose in mg/kg.
*Excluded from all pharmacokinetic parameter calculations and averages due to being an outlier.

### Summary of Results

In this study, the exposure of L-Dopa and its metabolite, dopamine, was evaluated following intranasal (IN) administration of a formulation (L-Dopa+arginine) in male Sprague-Dawley rats. Blood and brain tissue samples were collected up to 8 hours post-dose, and plasma and brain concentrations of L-Dopa and dopamine (some of which may be endogenous) were determined by LC-MS/MS. Pharmacokinetic parameters were determined using Phoenix WinNonlin (v8.0) software with or without sparse sampling.

Following the IN dosings of L-Dopa (2.4 and 3.6 mg/kg L-dopa)+10% Arginine respectively, detectable levels of dopamine were observed in the rat brain tissues. A significant increase in dopamine levels (155 ng/mL) at 1 hour was observed in the rat brain homogenates of the rats in Group (2) having been dosed with 3.6 mg/kg of L-Dopa with 10% arginine. It was also noted that the level of dopamine was detectable beyond 8 hours.

In the foregoing description, specific details are set forth, such as specific materials, processes parameters, etc., to provide a thorough understanding of the invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment and are non-limiting.

The invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A method of treating Parkinson's disease comprising administering to the olfactory region of the nose of a patient in need thereof a pharmaceutical composition comprising levodopa or a pharmaceutically acceptable salt thereof and a positively charged amino acid.

2. The method of claim 1, wherein the composition further comprises a nasally acceptable vehicle.

3. The method of claim 2, wherein the nasally acceptable vehicle is an aqueous solution, a suspension, an ointment, a cream, a gel, or a combination thereof.

4. The method of claim 2, wherein the levodopa or pharmaceutically acceptable salt thereof is dissolved or suspended in the nasally acceptable vehicle.

5. The method of claim 2, wherein the positively charged amino acid is dissolved or suspended in the nasally acceptable vehicle.

6. The method of claim 1, wherein the pharmaceutical composition comprises solid particles comprising the levodopa or pharmaceutically acceptable salt thereof and the positively charged amino acid.

7. The method of claim 1, wherein the positively charged amino acid is selected from lysine, arginine, histidine, and a combination thereof.

8. The method of claim 7, wherein the positively charged amino acid is arginine.

9. The method of claim 1, wherein the pharmaceutical composition does not comprise a decarboxylase inhibitor.

10. The method of claim 1, wherein the pH value of the pharmaceutical composition is from about 6 to about 9.

11-13. (canceled)

14. The method of claim 1, wherein the pharmaceutical composition contacts the olfactory nerves of the patient during administration.

15-16. (canceled)

17. The method of claim 16, wherein the concentration of levodopa or pharmaceutically acceptable salt thereof present in the pharmaceutical composition is from about 6 mg/mL to about 10 mg/mL.

18-19. (canceled)

20. The method of claim 1, wherein the ratio (w/w) of the positively charged amino acid to the levodopa or pharmaceutically acceptable salt thereof is greater than about 2:1; greater than about 5:1; greater than about 8:1; greater than about 10:1; or greater than about 12:1.

21. The method of claim 1, further comprising orally administering a second amount of levodopa or a pharmaceutically acceptable salt thereof and a decarboxylase inhibitor.

22. The method of claim 21, wherein the nasal administration is to treat off periods in the patient in need thereof associated with the oral administration.

23-25. (canceled)

26. The method of claim 1, wherein the ratio of the maximum concentration of dopamine in the brain to the maximum concentration of dopamine in the systemic plasma is greater than 10,000:1; greater than 1,000:1; greater than 10:1; greater than 50:1 or greater than about 100:1.

27-28. (canceled)

29. The method of claim 1, wherein in the pharmaceutical composition, the molar ratio of the positively charged amino acid to the levodopa or pharmaceutically acceptable salt thereof is greater than 2:1; greater than about 5:1 or greater than about 10:1.

30. The method of claim 1, wherein the pharmaceutical composition is administered from a nasal device adapted to deliver the composition to the olfactory region of the nose.

31-36. (canceled)

37. A system comprising a device adapted to deliver a payload to the olfactory region of a human nose and a nasal composition comprising levodopa or a pharmaceutically acceptable salt thereof, a positively charged amino acid and a pharmaceutically acceptable nasal vehicle.

38. A method of delivering levodopa or a pharmaceutically acceptable salt thereof to a patient identified as in need of levodopa therapy,
comprising administering to the olfactory region of the nose of the patient a pharmaceutical composition comprising levodopa or pharmaceutically acceptable salt thereof and a positively charged amino acid,
wherein said method selectively delivers a therapeutically effective amount of the levodopa or pharmaceutically acceptable salt thereof to the brain tissues of the patient.
